(19) United States
(12) Patent Application Publication
(10) Pub. No.: US 2013/0266979 A1
(43) Pub. Date: Oct. 10, 2013

(54) LAB-ON-A-CHIP DEVICE, FOR INSTANCE FOR USE OF THE ANALYSIS OF SEMEN

(71) Applicants: Loes Irene Segerink, Enschede (NL); Adrianus Joseph Sprenkels, Lelystad (NL); Johan Gerrit Bomer, Haaksbergen (NL); Albert van den Berg, Nijverdal (NL)

(72) Inventors: Loes Irene Segerink, Enschede (NL); Adrianus Joseph Sprenkels, Lelystad (NL); Johan Gerrit Bomer, Haaksbergen (NL); Albert van den Berg, Nijverdal (NL)

(73) Assignee: Stichting voor de Technische Wetenschappen, JP Utrecht (NL)

(21) Appl. No.: 13/810,969

(22) PCT Filed: Jul. 21, 2011
(86) PCT No.: PCT/NL2011/050529

§ 371 Date: Jun. 25, 2013

(30) Foreign Application Priority Data
Jul. 22, 2010 (EP) 10170399.9

(51) Int. Cl. G01N 33/487

(52) U.S. Cl. CPC G01N 33/48707
USPC 435/29; 435/287.1; 29/825

(57) ABSTRACT

The invention provides a lab-on-a-chip device comprising a micro channel for a fluid, wherein the micro channel comprises a circumferential channel wall, wherein the channel wall comprises a first electrode, a second electrode, and a floating third electrode. The device can be used in a method for the analysis of an analyte fluid comprising flowing the analyte fluid through the channel of the electronic lab-on-a-chip device by measuring an electric signal between the first and the second electrode, especially measuring the electrical impedance between the first electrode and the second electrode. This may be used for instance for the analyses of mammalian semen.

### FIELD OF THE INVENTION

The invention relates to a lab-on-a-chip device, an electronic lab-on-a-chip device comprising such lab-on-a-chip device, and to a method for the production of such lab-on-a-chip device. The invention further relates to the use of such lab-on-a-chip device as detection device and to a method for the analysis of an analyte fluid.

### BACKGROUND OF THE INVENTION

Micro-fluidic and micro-electronic devices are known in the art. US2010006441, for instance, describes a micro fluidic system comprising a space for containing a liquid and at least one lateral chamber in communication with said space, said lateral chamber containing a metal electrode. The lateral chamber and the space are designed to be filled by the same or different liquid when the system is active. Another example is described in WO2007107947, which describes designs of a microelectronic device comprising heating electrodes (HE) and field electrodes (FE) that have effect in the same sub-region of a sample chamber. By applying appropriate voltages to the field electrodes (FE), an electrical field (E) can be generated in the sample chamber. By applying appropriate currents to the heating electrodes (HE), the sample chamber can be heated according to a desired temperature profile. The heating electrodes (HE) may optionally be operated as field electrodes such that they generate an electrical field in the sample chamber, too.

Such devices may be used for different applications. Because of their small dimensions, they are sometimes also called lab-on-a-chip. For instance, liquids may be measured on their properties.

Lab-on-a-chip devices might also be used for analysis of semen. A first step in the treatment of a couple with an unfulfilled desire to have children is the assessment of the semen quality. One of the parameters assessed with a semen analysis is the spermatozoa concentration, whereby the generally accepted lower limit for fertile men is 20×10⁶ mL⁻¹. Visually counting the spermatozoa in semen by putting the semen into a counting chamber is the gold standard for this determination. This labour intensive method is replaced by a computer assisted semen analysis system in larger hospitals. The results of the manual test are often subjective and can hardly be compared between different laboratories, while the computer assisted semen analysis system is expensive and needs comprehensive quality control. In addition, only reliable results are obtained after analysis of at least three consecutive samples.

It is further noted, that WO2010000977 describes a method of measuring the flow rate of an electrically conductive liquid in laminar flow and to an implementation measurement device that are easy to implement, simple to produce and compact. For this purpose, the invention provides a device for measuring the flow rate, comprising: a channel; a pair of electrodes; and at least one device for measuring the voltage between the electrodes of said pair, an electrical double layer forming at the interface of each electrode with the conducting liquid. The device comprises means designed so that, when the conducting liquid is flowing through the channel, the flow velocity fields in the diffuse layers facing each electrode are different and so that the convective/diffusive charge equilibria of the electrical double layer at the interface of each electrode are different.

Further, WO2009126257 describes a method to manufacture microfluidic sensors, typically including componentizing substrate layers. One such method includes providing a plurality of layers of material configured to permit their stacking to form at least a first cap layer, a first channel layer, an interrogation layer, and a second channel layer. During assembly, ribbon sections of substrate layers are sandwiched to cooperatively align elements through-the-thickness of the sandwich. Individual sensors are then removed from the sandwich ribbon. A componentizing step includes forming one or more elements for successive sensors spaced along the axial length of a ribbon. Certain elements include electrically conductive patterned structures preferably printed onto a substrate using conductive ink and a printing process, sometimes placing material in operable position to conduct electricity through the thickness of at least one ribbon. Other elements may include channels, tunnels, and vias that can be machined, stamped, or cut into a ribbon section.

US20070240986 describes an electrochemical microfluidic device which has one or a plurality of microstructures, such as a micro channel, in which an electrically conductive means is integrated to reduce the ohmic resistance within the microstructure and hence to improve electrochemical measurements particularly when large current densities are involved. The electrically conductive means can be connected as a counter-electrode and can be used to re-generate the product of the reaction occurring at the working electrode. A method of fabricating electrochemical microfluidic devices comprising such an electrically conductive means is also described. US20070240986 indicates that it may particularly be used in all electrochemical sensor applications where detection is performed in small volumes.

Further, U.S. Pat. No. 6,482,306 describes an electroosmotic mixing device and a method for mixing one or more fluids for use in meso- or microfluidic device applications. The mixing device provides batch or continuous mixing of one or more fluids in meso- or microfluidic channels. An electric field is generated in the channel in substantial contact with chargeable surfaces therein. No alterations of the geometry of existing flow paths need be made, and the degree of mixing in the device can be controlled by the length of the electrodes, the flow rate past the electrodes, and the voltage applied to those electrodes. The degree of mixing is affected by choice of materials for the chargeable surface (in some cases by the selection of materials or coatings for channel walls) and the ionic strength of the fluids and the type and concentration of ions in the fluids. The ionic strength of fluids to be mixed is sufficiently low to allow electro osmotic flow. The method and device of U.S. Pat. No. 6,482,306 may be applied to fluids to having low ionic strength less than or equal to about 1 mM.

WO2005093009 describes an etched dielectric film for use in microfluidic devices. Channels, recesses, and other features can be etched into the films to make them suitable for use in microfluidic devices.

### SUMMARY OF THE INVENTION

Important quality parameters for semen are for instance the concentration, motility and the morphology. With a micro fluidic chip it was shown that it is possible to determine the spermatozoa concentration using electrical impedance measurements. This micro fluidic chip consisted of a micro channel in which the electrodes are placed in a planar configuration on the same side of the channel and the electrical impedance is measured between these electrodes. A particle or cell passing the electrodes causes a change in the impedance. This change may be size dependent at measurement frequencies below 1-3 MHz. With the micro fluidic chip it was possible to distinguish 6 μm polystyrene beads, spermatozoa and HL-60 (Human promyelocytic leukaemia cells) cells. However the deviations in the changes for each particle type were relatively large, possibly due to the inhomogeneous electrical field between the two planar electrodes.

To improve this, the use of parallel electrodes instead of planar electrodes was investigated. The advantage of planar electrodes is that it may be easier to fabricate the device. However simulations showed that planar electrodes yield a smaller impedance change than parallel ones when a cell passes the electrodes. This can be caused by the less homogenous electrical field in case of planar electrodes. Furthermore the change in impedance that is measured in the planar electrode configuration depends on the position of the particle or cell in the channel. Parallel electrodes do not have this dependency since the field is more homogenous. However, the fabrication of parallel electrodes is difficult. Making electrical contact with the electrodes at the other side of the channel and alignment of the electrodes to each other is necessary, increasing the complexity of the fabrication considerably.

Hence, it is an aspect of the invention to provide an alternative lab-on-a-chip device, which preferably further at least partly obviates one or more of above-described drawbacks. Especially, it is an aspect of the invention to provide a lab-on-a-chip device that may be easier to produce and that may produce a good and reliable signal when an analyte passes through the lab-on-a-chip device.

The invention improves the fabrication of a lab-on-a-chip device with parallel electrodes, by putting (in an embodiment) a floating electrode at the other side of the channel. This makes the fabrication process easier and it is possible to make wafers, such as glass/glass chips, without a necessary additional layer in between. Due to the floating electrode, there may exist no problems with the connection of the electrodes, since the two electrodes that need to be connected are on the same side of the channel.

In a first aspect, the invention provides a lab-on-a-chip device comprising a micro channel (“channel”) for a fluid, wherein the micro channel comprises a circumferential channel wall (“channel wall”), wherein the channel wall comprises a first electrode, a second electrode, and a floating third electrode (“floating electrode”).

Such lab-on-a-chip device may relatively easy be made, especially in the sense of applying the electrodes and aligning parts together (see also below). Further, due to the presence of the floating electrode (the “third electrode”), relative homogeneous electric fields may be created, which may facilitate production of a good and reliable signal.

The term lab-on-a-chip device relates to a device, such as a glass or silicon wafer based device, with one or more micro channels. Further, such device in general comprises facilities to measure or influence the material within the channel(s). Here, the lab-on-a-chip device comprises at least two electrodes, the first and the second electrode, and the floating electrode. Herein, the floating electrode is configured as floating electrode for both the first electrode and the second electrode. Herein, the term “floating” relates to the fact that the electrode is electrically floating. The electrode itself is integrated in the device.

The first electrode, second electrode, and floating electrode, are preferably configured and arranged in such a way, that the first electrode and the floating electrode, and the second electrode and the floating electrode, allow generation of a homogeneous electric field between the first electrode and the floating electrode, and the second electrode and the floating electrode, respectively. For instance, this may be achieved by arranging the first electrode and the floating electrode opposite of each other, and by arranging the second electrode and the floating electrode opposite of each other. Hence, in a specific embodiment, the first electrode and the second electrode are arranged opposite of the floating third electrode. When the electrodes are arranged opposite of each other, the electrodes are facing each other, or at least part of the electrodes are facing each other. The parts that are directly opposite of each other have an “overlapping” area. The term “overlapping” does thus not indicate being in contact with each other.

The term “first electrode” may refer to a plurality of electrically conductively interconnected first electrodes. The term “second electrode” may refer to a plurality of electrically conductively interconnected second electrodes. The term “floating electrode” may refer to a plurality of (electrically conductively interconnected) floating electrodes. The first electrode and/or the second electrode (and also the floating electrode) may in principle have any shape. For instance, in an embodiment, the first electrode has an intended structure, such as a kind of hand with fingers shape. Likewise, this may apply to the second electrode. In an embodiment, the first electrode and the second electrode are configured as interdigitated electrodes.

The term “micro channel” especially refers to channels having cross-sectional dimensions preferably in the range of about 0.1-1000 μm. The term “cross-sectional dimension” may relate to height, width and in principle also to diameter. When a wall (including bottom or top of the channel) of the channel is irregular or curved, the terms “height” and “width” may also relate to mean height and mean width, respectively.

Especially, at locations of the first and the second electrode the micro channel has a channel width and channel height in the range of 0.1-500 μm, especially in the range of 5-50 μm, more especially, at the locations of the first and the second electrode the micro channel has a channel width in the range of 1-500 μm and a channel height in the range of 0.1-300 μm, especially a channel width and channel height in the range of 2-200 μm. The phrase “at locations of the first electrode” and similar phrases indicate that a cross-section of the channel where the first electrode is located, has the indicated dimension(s). Upstream or downstream thereof, the channel dimension(s) may differ, and may for instance be in the range of 0.1 μm-5 mm. The channel dimensions may also substantially be constant, and for instance be in the range of 0.1-500 μm (as indicated above), over substantially the entire length of the channel.

It is preferred that the impedance of the reference is minimized, and a signal due to the analyte particle when at the location of the reference electrode is substantially (reduced). To achieve this, it is possible to create different electrode areas, such as a relatively large electrode surface in the second channel. This may reduce resistance and thus also impedance. Therefore, in a specific embodiment, the first electrode in the first micro channel has a first electrode surface area, the second electrode in the second micro channel has a second electrode surface area, the floating electrode has in the first micro channel a floating electrode first surface area and in the second micro channel a floating electrode second surface area, and one or more of (1) the second electrode surface area is larger than the first electrode surface area, and (2) the floating electrode second surface area is larger than the floating electrode first surface area, applies. Preferably, the surface area of the second electrode and/or the floating electrode in the second channel is 1.5 times larger, more preferably 2 times larger, and especially 5 times larger, such as 10 times larger, than the area of the first electrode and/or of the floating electrode in the first channel, respectively. For instance, the ratio may be even 20 or larger, or even 100 or larger.

Especially, the first electrode is arranged opposite of the floating electrode and the second electrode is arranged opposite of the floating electrode. Assuming a rectangular cross-section, like a square cross-section, at one side of the micro channel the first electrode may be arranged, and the floating electrode may be arranged at an opposite side, at a distance substantially equal or in the range of the height of the micro channel. Likewise, assuming a rectangular cross-section, like a square cross-section, at one side of the (second) micro channel the second electrode may be arranged, and the floating electrode may be arranged at an opposite side, at a distance substantially equal or in the range of the height of the micro channel. Hence, there will be overlapping surface areas, where the first electrode and the floating electrode and where the second electrode and the floating electrode are facing each other. Preferably, the overlapping area of the second electrode and the floating electrode is 1.5 times larger, more preferably 2 times larger, and especially 5 times larger, such as 10 times larger, especially at least 20 times larger, than the overlapping area of the first electrode and the floating electrode.

This may apply to both the single channel variant as the dual channel variant (see also below).

Hence, in a specific embodiment, the overlapping area of the second electrode and the floating electrode (in the second micro channel) is 1.5 times larger, etc., than the overlapping area of the first electrode and the floating electrode (in the first micro channel). Such configurations may reduce counting particles twice. Therefore, in an embodiment the first electrode and the floating electrode have an overlapping area where they are facing each other, the second electrode and the floating electrode have an overlapping area where they are facing each other, and the overlapping area between the second electrode and the floating electrode is at least 1.5 times larger, more preferably 2 times larger, and especially 5 times larger, such as 10 times larger, especially at least 20 times larger than the overlapping area between the first electrode and the floating electrode.

The second electrode (in the second micro channel) may thus be used as reference electrode.

### Two or More Channel Embodiments

The lab-on-a-chip device comprises at least one channel (sometimes also indicated as “first channel”). This channel may be curved or straight, or may comprise curved parts and straight parts. When the first and the second electrode are arranged in one single channel, this may lead to a double counting of the analyte. When the device comprises a single channel with two electrode pairs (i.e. first electrode and floating electrode and second electrode and floating electrode), this may in some embodiments be less preferred. It is less desirable to have these electrode pairs in the channel with the cells or particles, since there is a probability that particles are simultaneously between both electrode pairs. Besides that the particles may be counted twice (in an analysis method embodiment), when flowing through the channel.

To solve this, the device may be equipped with two (or more) channels. For instance, in a specific embodiment one channel (“first channel”) may be configured to contain the analyte fluid, while the other channel (“second channel”) may be configured to contain a reference fluid (i.e. without the analyte), such as a reference liquid. In a specific embodiment, the lab-on-a-chip device comprises two or more channels. With more than one channel, a second or further channel (if applicable) may be used as reference channel. Hence, the micro channel comprises in such embodiments a first micro channel and a second micro channel. In some embodiments, this may be a single channel that branches in two channels in the device (and optionally downstream joins again), or it may be a device with two entirely separate channels.

Hence, in a specific embodiment, the lab-on-a-chip device comprises a first micro channel and a second micro channel, wherein the first micro channel comprises a circumferential first channel wall and wherein the second micro channel comprises a circumferential second channel wall, wherein the first channel wall comprises the first electrode, wherein the second channel wall comprises the second electrode, and wherein both the first channel wall and the second channel wall comprise the floating third electrode. In such embodiment, the floating third electrode is arranged and configured in such a way that the floating electrode is both present in the first channel and the second channel. For instance, in such embodiment the floating electrode may comprise two or more floating electrodes, preferably electrically conductively connected to each other, wherein a first floating electrode is comprised by the first channel wall and the second floating electrode is comprised by the second channel wall. However, in another embodiment, the lab-on-a-chip device comprises a first micro channel and a second micro channel, wherein the first micro channel comprises a circumferential first channel wall and wherein the second micro channel comprises a circumferential second channel wall, wherein the first channel wall comprises the first electrode, wherein the second channel wall comprises the second electrode, and wherein both the first channel wall and the second channel wall comprise a floating third electrode, wherein the (first) floating electrode in the first channel may (see above) or may not electrically be connected with the (second) floating electrode in the second channel.

Especially when the second channel is used as channel to contain a reference fluid, the first and the second channel may not be in fluid connection with each other. Hence, in an embodiment, the first micro channel and the second micro channel are not in fluid connection which each other. In another embodiment, the first and second channels are in fluid connection with each other, but an analyte is prevented to enter the second channel. In this way, the fluid in the second channel may be substantially identical to the fluid in the first channel, except for the presence of an analyte. Hence, in another embodiment, the first micro channel and the second micro channel are in fluid connection which each other, and wherein a fluid permeable membrane is arranged between the first micro channel and the second micro channel. Therefore, between the first channel and the second channel an analyte barrier may be arranged. In an embodiment, this may be a membrane, and in a more rigorous embodiment, the channels are not connected at all (i.e. it is not intended that liquid flows from one channel into the other).

In a specific embodiment, the floating electrode is comprised by the first channel and the second channel. Also part of the floating electrode may be arranged in between the channels (to ensure electrical conductivity between the part of the floating electrode in the first channel (first floating electrode) and the part of the floating electrode in the second channel (second floating electrode); as indicated above, in a specific embodiment, the first floating electrode and the second floating electrode are electrically connected). Hence, an interconnecting channel may be necessary to host at least part of the floating electrode. This interconnecting channel may thus interconnecting the first and the second channel. Since a fluid connection is in general not desired, the channel may be configured in such a way, that no fluid may migrate through the interconnecting channel. Hence, in a specific embodiment, the invention provides an embodiment of the lab-on-a-chip device wherein the first micro channel and the second micro channel are interconnected by an interconnecting channel, wherein the interconnecting channel is configured to host at least part of the floating third electrode, and wherein the interconnecting channel further comprises a fluid barrier. This fluid barrier may for instance be a hardened resin. When two non-electrically connected floating electrodes are applied, the interconnecting channel may be absent.

Especially when the second channel is applied as reference channel, it is desired that this channel does not substantially influence the process in the first channel or the measurement, for instance an impedance measurement. Assuming for instance an impedance measurement, it is preferred that the impedance of the reference channel is minimized. For instance, the reference channel might be filled with a high ionic liquid, such as a salt solution. It is additionally or alternatively also possible to create a large electrode surface in the second channel. This may reduce resistance and thus also impedance. Therefore, in a specific embodiment, the first electrode in the first micro channel has a first electrode surface area, the second electrode in the second micro channel has a second electrode surface area, the floating electrode has in the first micro channel a floating electrode first surface area and in the second micro channel a floating electrode second surface area, and one or more of (1) the second electrode surface area is larger than the first electrode surface area, and (2) the floating electrode second surface area is larger than the floating electrode first surface area, applies. Preferably, the surface area of the second electrode and/or the floating electrode in the second channel is 1.5 times larger, more preferably 2 times larger, and especially 5 times larger, such as 10 times larger, than the area of the first electrode and/or of the floating electrode in the first channel, respectively. For instance, the ratio may be even 20 or larger, or even 100 or larger.

### Application

The lab-on-a-chip device can be used in and for several applications. For instance, it can be used to control reactions within the channel(s). It may also be used for analysis of fluids. For instance, the fluid can be a gas or a liquid, especially a liquid. The term fluid may also relate to a mixture of fluids, such as a mixture of liquids.

The analyte fluid (“fluid”) may be any fluid in which an analyte may be present. Especially, it may be a fluid wherein the analyte may be dispersed or float, like dust particles in air. In a specific embodiment, the analyte fluid is a liquid, such as water. In yet another embodiment, the analyte fluid is a gas. In an embodiment, the analyte fluid comprises semen. The analyte may be any particle, but may especially be selected from the group consisting of a virus, a bacterium, and a cell. In a specific embodiment, the analyte comprises a sperm cell, such as a spermatozoon. The term “analyte” may also relate to a plurality of analytes. It may also refer to a plurality of different analytes.

Hence, in an embodiment, the (electronic) lab-on-a-chip device may be used for the analysis of (mammalian) semen, such as human or animal semen. For instance, when applying electrical impedance, semen may be analyzed on the presence, and concentration of the sperm cells, such as spermatozoon. The spermatozoon may also be analysed on for instance morphology. The (electronic) lab-on-a-chip device may be used for the analysis of liquids comprising particles, such as for the analysis of blood (counting or estimating red blood cells RBC's concentration).

The (electronic) lab-on-a-chip device may be used for electrical impedance measurements in for instance electrophoresis experiments.

### Electronic Lab-on-a-Chip Device

As mentioned above, the lab-on-a-chip device may be used in several applications. To this end, the lab-on-a-chip device will in general be connected to an electronic device. Hence, in a further aspect, the invention provides an electronic lab-on-a-chip device comprising the lab-on-a-chip device as defined herein, and an electronic device electronically connected with the first and the second electrode.

In an embodiment, the electronic device may be selected from the group consisting of a current meter, a voltmeter, a current generator (such as an AC or DC or AC+DC current generator), a voltage source (such as an AC or DC or AC+DC voltage source), and an electrical impedance meter. The electronic device may especially comprise an electrical impedance meter configured to measure the electrical impedance between the first electrode and the second electrode.

### Analysis Method

Hence, in a further aspect the invention provides a method for the analysis of an analyte fluid comprising flowing the analyte fluid through the channel of the electronic lab-on-a-chip device as defined herein, and measuring an electric signal between the first and the second electrode, especially measuring the electrical impedance between the first electrode and the second electrode.

The method may thus comprise flowing the analyte fluid through the first channel and measuring the electrical impedance between the first electrode and the second electrode. The method may further comprise using a device with a first and a second channel, as defined herein, and providing a reference fluid in the second channel. This reference fluid may be a completely other fluid than the first fluid, but may in an embodiment also be a filtered first fluid, for instance filtered through a membrane. Such method may for instance be used to analyse semen on fertility of the spermatozoon, as indicated above.

### Production Method

As indicated above, the lab-on-a-chip device may be produced in a relatively simple way, providing combining both the ease of applying planar electrodes and the relative simple application of the floating electrode (oppositely arranged of the first and the second electrode), thereby obtaining parallel electrodes.

In a further aspect, the invention therefore provides a method for the production of a lab-on-a-chip device as defined herein, comprising:

a. providing a first wafer, with a first first wafer side (“top side of the first wafer”) and an opposite second first wafer side (“back side of the first wafer”), and a second wafer, with a first second wafer side (“top side of the second wafer”) and an opposite second second wafer side (“back side of the second wafer”);

b. creating a micro channel structure in the first first wafer side;

c. providing a floating electrode to a wall of the micro channel structure;

d. creating at least a first access channel and a second access channel between the micro channel structure and the second first wafer side;

e. providing a first electrode and a second electrode to the first second wafer side;

f. attaching the first first wafer side and the first second wafer side to each other with the first electrode and second electrode comprised by the micro channel structure.

The wafer may for instance be selected from the group consisting of a glass wafer and a silicon wafer.

The creation of the micro channel structure in general includes etching, such as isotropic or anisotropic etching. Especially, photo lithography may be applied.

The term “micro channel structure” herein refers to a structure comprising at least one channel (first channel), and optionally more than one channel (first and second channel, and optionally more channels.

Electrodes may be applied to the wafer(s) by sputtering of conductive metals, especially by Pt sputtering.

The phrase “attaching the first first wafer side and the first second wafer side to each other with the first electrode and second electrode comprised by the micro channel structure”, especially includes that by arranging the first wafer and the second wafer to each other, a substantial part of the micro channel structure on the first wafer is closed with the second wafer, in such a way, that the resulting channel(s) contain the first electrode, the second electrode and the floating electrode. The above method is especially suited to create oppositely arranged electrodes, i.e. the first electrode being arranged opposite of the floating electrode and the second electrode being arranged opposite of the floating electrode. In fact, the second wafer can be considered as closure (see also embodiments below).

In a specific embodiment, (b) creating a micro channel structure in the first first wafer side includes creating at least a first micro channel, creating a second micro channel and creating an interconnecting channel interconnecting the first micro channel and the second micro channel. In a further embodiment, (c) providing a floating electrode to a wall of the micro channel structure includes providing the floating electrode to a wall of the first micro channel, to a wall of the interconnecting channel, and to a wall of the second micro channel. Especially in the latter embodiment, method part (f) further includes arranging a fluid barrier in the interconnecting channel.

Such fluid barrier may be a resin that after introduction is hardened. However, it may also comprise other materials, such as a silicone sealant. For instance, a UV curing glue may be applied.

In this way, the method may provide an embodiment of the lab-on-a-chip device such as defined herein, wherein the device comprises a layered structure comprising a first wafer, comprising the micro channel, and comprising the floating electrode, and a second wafer, comprising the first electrode and the second electrode, wherein the first wafer and the second wafer are configured relative to each other to provide the micro channel with the circumferential channel wall, wherein the channel wall comprises the first electrode, the second electrode, and the floating third electrode.

In a further aspect, the invention provides a method for the production of a lab-on-a-chip device, such as defined herein, the method comprising:

a. providing a first wafer, with a first first wafer side and an opposite second first wafer side, and a closure (such as a second wafer), with a first closure side and an opposite second closure side, wherein the first closure side comprises a floating electrode;

b. creating at least a first access channel and a second access channel between the first closure side and the opposite second closure side;

c. providing a first electrode and a second electrode to the first second wafer side;

d. providing an intermediate layer to the first second wafer side with first electrode and second electrode;

e. creating a micro channel structure in the intermediate layer, with the first electrode and the second electrode comprised by the micro channel structure;

f. providing the intermediate layer and the first closure side to each other with the floating electrode comprised by the micro channel structure.

Especially with such method, an embodiment of the lab-on-a-chip device may be provided, such as defined herein, wherein the device comprises a layered structure comprising a first wafer, comprising the floating electrode, an intermediate layer comprising the micro channel, and a closure, especially a second wafer, comprising the first electrode and the second electrode, wherein the first wafer, the intermediate layer and the closure, especially the second wafer, are configured relative to each other to provide the micro channel with the circumferential channel wall, wherein the channel wall comprises the first electrode, the second electrode, and the floating third electrode. The closure comprises at the first closure side the floating electrode. For instance, a wafer with floating electrode may be used. In an embodiment, the closer substantially consists of the floating electrode. For instance, the closure may be a conductive silicon wafer, such as a highly doped silicon wafer. In another embodiment, the closure is a metallic closure.

Further, the method may include creating a plurality of access channels, for instance an inlet access channel and an outlet access channel for the first channel, and optionally an inlet access channel and an outlet access channel for the second channel. Further, the method may include creating an access channel in the first wafer, configured to provide access to the interconnecting channel when the first and second wafers are arranged to each other. Alternatively or additionally, the method may include creating an access channel in the second wafer, configured to provide access to the interconnecting channel when the first and second wafer is arranged to each other.

The terms “upstream” and “downstream” relate to an arrangement of items or features relative to the propagation of the fluid flow, wherein relative to a first position within a flowing fluid, a second position in flowing fluid closer to the source of the flowing fluid is “upstream”, and a third position within the flowing fluid further away from the source of the flowing fluid is “downstream”.

The term “substantially” herein, such as in “substantially all emission” or in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices herein are amongst others described during operation. As will be clear to the person skilled in the art, the invention is not limited to methods of operation or devices in operation.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a-1i (cross-sections) show schematically the fabrication process. A wafer is firstly sputtered with Cr and Au layers. By means of photolithography (and etching), the micro fluidic channel is formed. After that a shadow mask is used for the local sputtering of the floating electrode. After removing several layers, access holes are powder blasted (see for instance Schlautmann et al., J. Micromechanics and Microengineering, vol. 11 (4) 2001, 386-389) from the backside. On another wafer, the embedded connecting electrodes are formed using a lift-off technique. After that both glass wafers were bounded together;

FIGS. 2a-2e (cross-sections) schematically depict some embodiments of the lab-on-a-chip device;

FIGS. 3a-3b (cross-sections) schematically depict another embodiment of the lab-on-a-chip device;

FIG. 4 shows the electrical characterisation of the lab-on-a-chip device;

FIG. 5 shows a schematic diagram of the measurement set-up. The lab-on-a-chip device holder makes fluidic and electrical connection to the chip. In this diagram, the inlet and outlet are located underneath the middle two screw threads and the electrodes of the lab-on-a-chip device are connected to the middle four electrodes on the lab-on-a-chip device holder. So each electrode on the micro fluidic lab-on-a-chip device has two electrical connections on the holder. Input 1 (ip1) of the HF2IS measures the voltage over the chip, while input 2 (ip2) measures indirectly the current through the lab-on-a-chip device;

FIG. 6 shows a typical example of an electrical impedance measurement where four beads passed the electrode pair;

FIGS. 7a-7c schematically depict part of an alternative fabrication process;

FIGS. 8a-8b schematically depict a further embodiment according to the invention;

FIGS. 9a-9b schematically depict further embodiments according to the invention.

### DESCRIPTION OF PREFERRED EMBODIMENTS

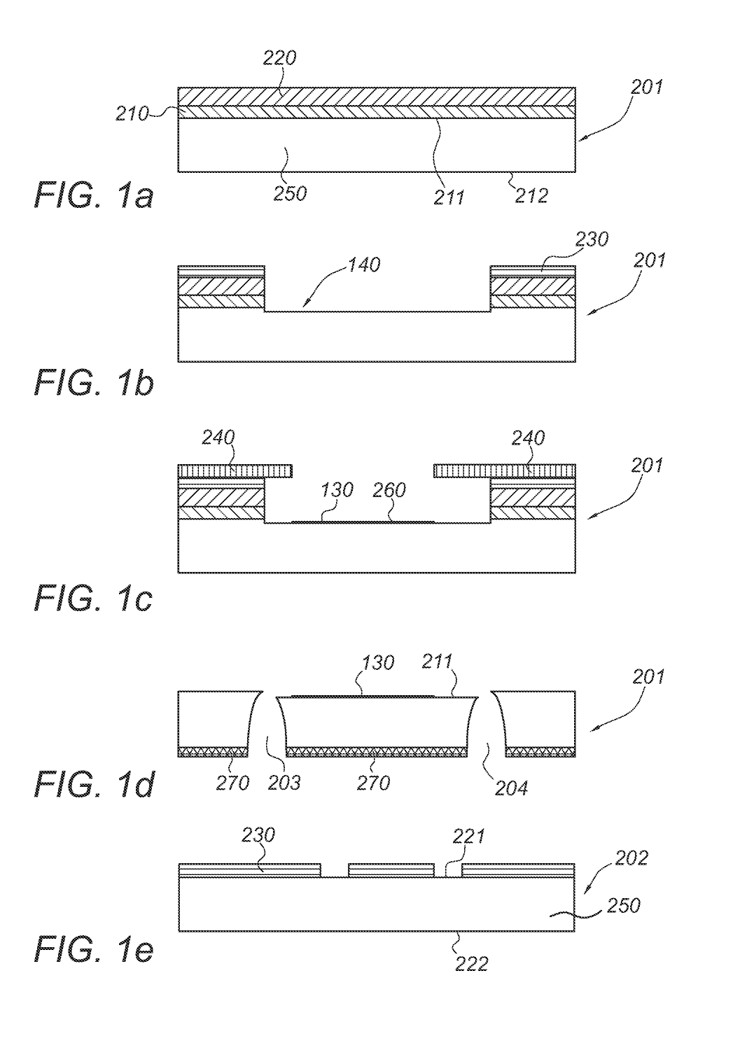

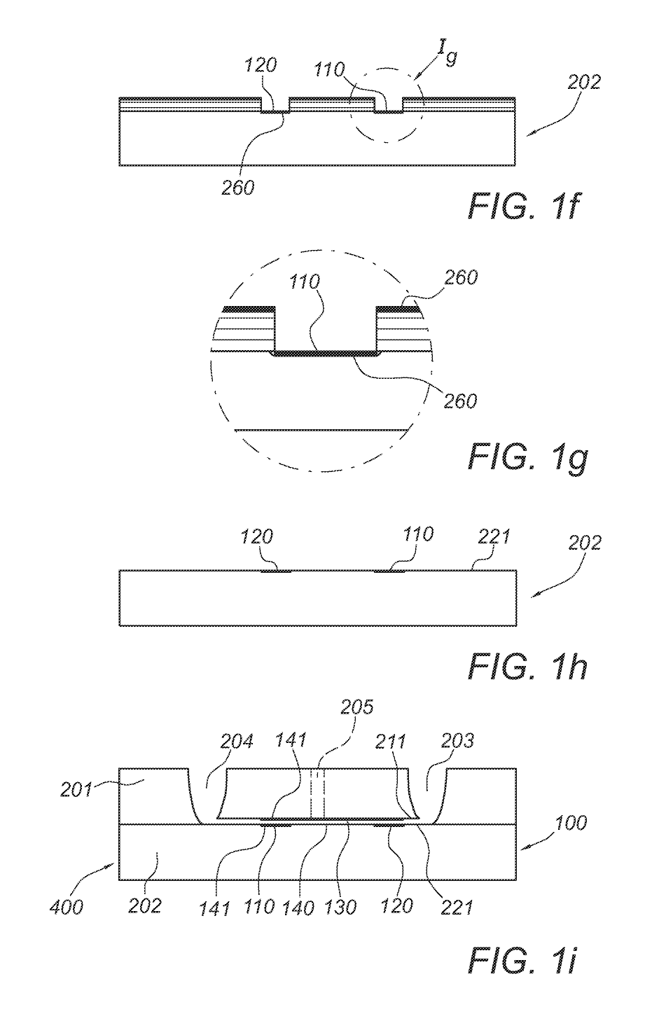

FIGS. 1a-1i shows a schematic diagram of an embodiment of the fabrication process.

First wafer 201, such as a glass wafer, is firstly sputtered with Cr and Au layers (a1); the Cr-layer is indicated with reference 210; the Au-layer is indicated with reference 220. This is applied on the first first wafer side or top surface 211 of the first wafer 201. By means of photolithography (and etching), a channel structure 140 is formed, with reference 230 indicating a photo resist. After that a shadow mask 240 is used for the sputtering of a floating electrode 130; especially a metal such as Pt can be applied. After removing several layers, access holes (and optionally a barrier access channel, see below), here first access channel 203 and second access channel 204, are powder blasted from the backside, i.e. from the second first wafer side or back side 212 of the first wafer 201, with reference 270 indicating a photo-patternable foil. FIG. 1i schematically depicts an embodiment wherein a barrier access channel 205 may be present in the first wafer 201 (this may lead to (small) hole in the floating electrode 130; see also below).

On second wafer 202, for instance also a glass wafer, embedded connecting electrodes are formed using a lift-off technique: a photo resist 230 is applied, with photolithography photo resist 230 at the future positions of the electrodes is etched away, the first electrode 110 and second electrode 120 are provided by sputtering a metal (especially Pt), and then photo resist (with surplus Pt) is removed. This is applied on the first second wafer or top side 221 of the second wafer 202. The opposite side (second second wafer side or bottom side of the second wafer 202) of the second wafer is indicated with reference 222.

In a specific embodiment, the first electrode 110 and the second electrode 120 have a shortest distance of at least 0.2 mm, preferably at least 0.5 mm

Having produced the first wafer 201 with channel structure 140 and floating electrode 130, and having produced the second wafer 202 with first electrode 110 and second electrode 120, the wafers can be attached to each other, for instance via direct bonding (such as fusion bonding). In this way, lab-on-a-chip device 100 is obtained.

The first and second electrodes 110,120 may thus be on the surface 221 of the second wafer, but may preferably, as indicated above (see enlargement 1g), be embedded. In such embodiment, the depth of the part of the second wafer 202 etched away (b1) is substantially equal to the height of the later provided first and second electrodes 110,120.

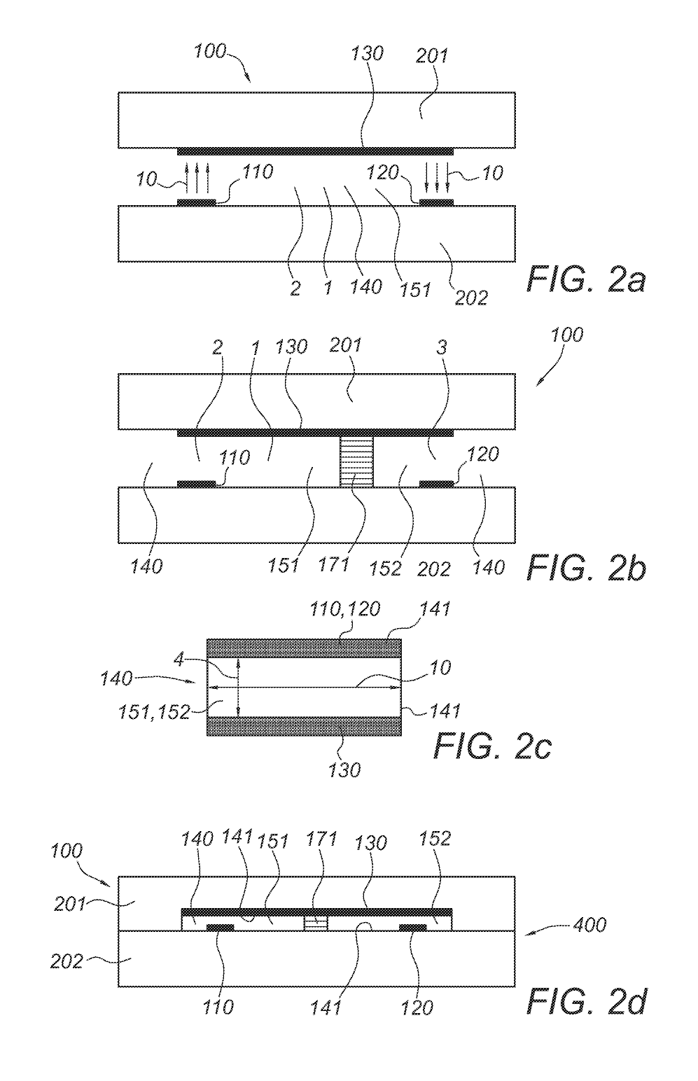

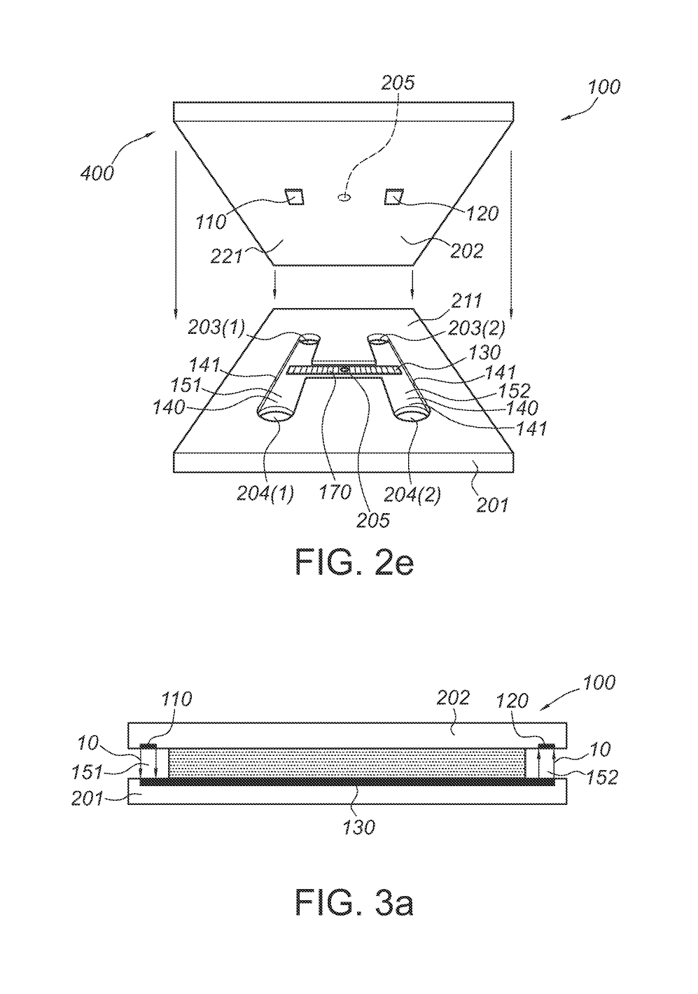

Electrode material, preferably Pt, is indicated with reference 260; wafer material, such as glass, is indicated with reference 250. When a voltage would be applied to the first and second electrodes 110,120, an electrical field 10 is generated, for instance as depicted in FIG. 2a.

FIG. 2a schematically depicts in more detail a cross-section of a variant of the lab-on-a-chip device 100 obtained at (c) in FIG. 1. Fluid 1 may flow through the micro channel structure 140. Reference 2 schematically depicts an analyte, such as a particle. Assuming a flow from left to right, the first electrode 110 is arranged upstream of the second electrode 120.

FIG. 2b schematically depicts in more detail a cross-section of a variant of the lab-on-a-chip device 100 obtained at (c) in FIG. 1, wherein a fluid barrier 171 is arranged between the first electrode 110 and the second electrode 120. For instance, this may be applied to create two channels. References 151,152 indicate a first and a second channel, respectively. For instance, in such variant, the first channel 151 may be used to flow the fluid 1, for analysis or treatment, etc., and the second channel 152 may contain a reference fluid 3. As can be seen in this figure, the floating electrode 130 is configured to be arranged in both channels.

FIG. 2c schematically depicts an embodiment of a channel within the channel structure 140, for instance first channel 151 or second channel 152. The height of the channel is indicated with h and the width is indicated with w. In principle, the circumferential wall 141 (including side walls, bottom and top), may be curved. For instance, the side walls may be curved when isotropic etching is applied. In such instance, the mean width (or mean height) is used as width w (or height h).

FIG. 2d schematically depicts in detail an implementation as chip, i.e. as a lab-on-a-chip device 100, with first wafer 201 attached to second wafer 202, thereby creating a channel structure 140, wherein within the channel(s) (151/152) of the channel structure 140 the first electrode 110, the second electrode 120 and the floating electrode 130 are arranged.

FIG. 2e schematically depicts an embodiment of the lab-on-a-chip device 100, which may be the same lab-on-a-chip device 100 as schematically depicted in FIGS. 2b and 2c. By way of example, the first wafer 201 and the second wafer 202 are opened. The first electrode 110 and second electrode 120 are comprised by the top side 221 of the second wafer 202. Further, second wafer 202 comprises optionally a barrier access channel 205. This channel 205 is configured to allow access to an interconnecting channel 170. Preferably, however, the barrier access channel 205 is provided in the first wafer 201. This may lead to a (small) hole in the floating electrode 130. In the schematic drawing, the first micro channel 110 and the second micro channel 120 are interconnected by interconnecting channel 170. The interconnecting channel 170 is configured to host at least part of the floating third electrode 130. In an embodiment, through barrier access channel 205, fluid barrier 171 may be introduced. For instance, a resin may be introduced, which is hardened after introduction into the interconnecting channel 170. In this way, the interconnecting channel 170 may further comprise fluid barrier 171. This fluid barrier 171 substantially blocks migration of fluid or components in the fluid from one side of the fluid barrier 171 to the other side of the fluid barrier 171 (here from the first channel 151 to the second channel 152 and vice versa). Further, the first wafer 201 comprises a first access channel 203 for the first channel 151, here indicated as first access channel 203(1), for instance configured to allow introduction of the fluid 1. Further, the first wafer 201 comprises a second access channel 204 for the first channel 151, here indicated as second access channel 204(1), for instance configured to allow exhaust of the fluid 1. In this embodiment, likewise, the first wafer 201 comprises a first access channel 203 for the second channel 152, here indicated as first access channel 203(2), for instance configured to allow introduction of the reference fluid 3. Further, the first wafer 201 comprises a second access channel 204 for the second channel 152, here indicated as second access channel 204(2), for instance configured to allow exhaust of the reference fluid 3. Assuming fluid(s) flowing from the first access channel(s) to the second access channel(s) (respectively), the first access channel is upstream, and the second access channel is downstream of the first electrode (and/) or second electrode. The lab-on-a-chip device 100 thus comprises a plurality of access channels.

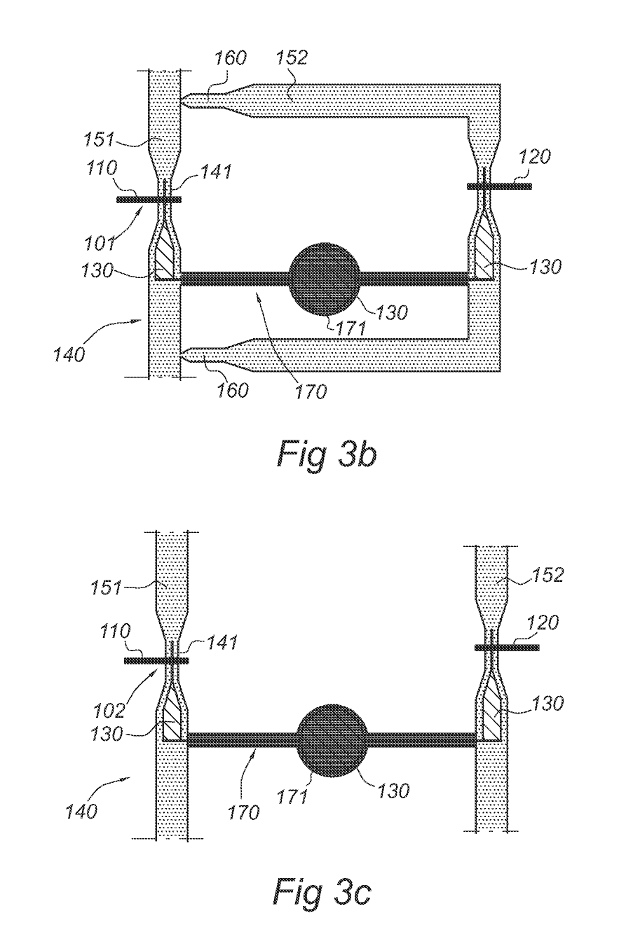

FIG. 3a schematically depicts a cross-sectional side view, with by way of example an electrical field 10 between the first electrode 110 and floating electrode 130 and floating electrode 130 and second electrode 120.

In another embodiment, referring to FIG. 3a, the wafer 201, like a Si wafer, or a substantial part thereof, may be used as floating electrode 130. In such embodiment, a separate floating electrode in addition to the wafer may not be necessary anymore.

FIGS. 3b-3c schematically depict cross-sections of embodiments in the plane of the lab-on-a-chip device 100.

In FIG. 3b, the second channel 152 is a side channel of the first channel 151. In an embodiment, it is prevented that analytes migrate from the first channel 151 to the second channel 152. This can be achieved by arranging one or more filters 160 in the second channel 152. At least at an upstream part (at least upstream from the second electrode 120 in the second channel 152) such filter 160 may have to be arranged. In FIG. 3b, the second channel 152 joins the first channel again. Hence, in such embodiment, also in a downstream part (at least downstream from the second electrode 120 in the second channel 152), such filter 160 may have to be arranged. Note that in this embodiment, two access channels (not depicted), may suffice (one upstream of the branch (of second channel into first channel and second channel); and one downstream of the merge (of second channel into first channel)). Optionally, the second channel 152 does not join again the first channel 151 (in such embodiment 3 access channels may suffice (one upstream of the branch, and one at each end of the first channel and second channel, respectively). Filter 160 may be used as embodiment of an analyte barrier.

FIG. 3c schematically depicts an embodiment that may correspond to the embodiment schematically depicted in FIG. 2e.

FIG. 3b schematically depicts a configuration 1A (see table below) and FIG. 3c schematically depicts a 1B configuration (see table below).

FIGS. 2e and 3a-3c show embodiments wherein the floating electrode 130 is both present in the first channel 151 and the second channel 152. Alternatively, one may phrase that a first floating electrode is arranged in the first channel and a second floating electrode is arranged in the second channel, and the first floating electrode and the second electrode are electrically conductively connected (and can thus be considered as one floating electrode).

Further, in nearly all FIGS. 1a-3c, configurations are shown wherein the first electrode 110 is arranged opposite of the floating electrode 130 and the second electrode 120 is arranged opposite of the floating electrode 130.

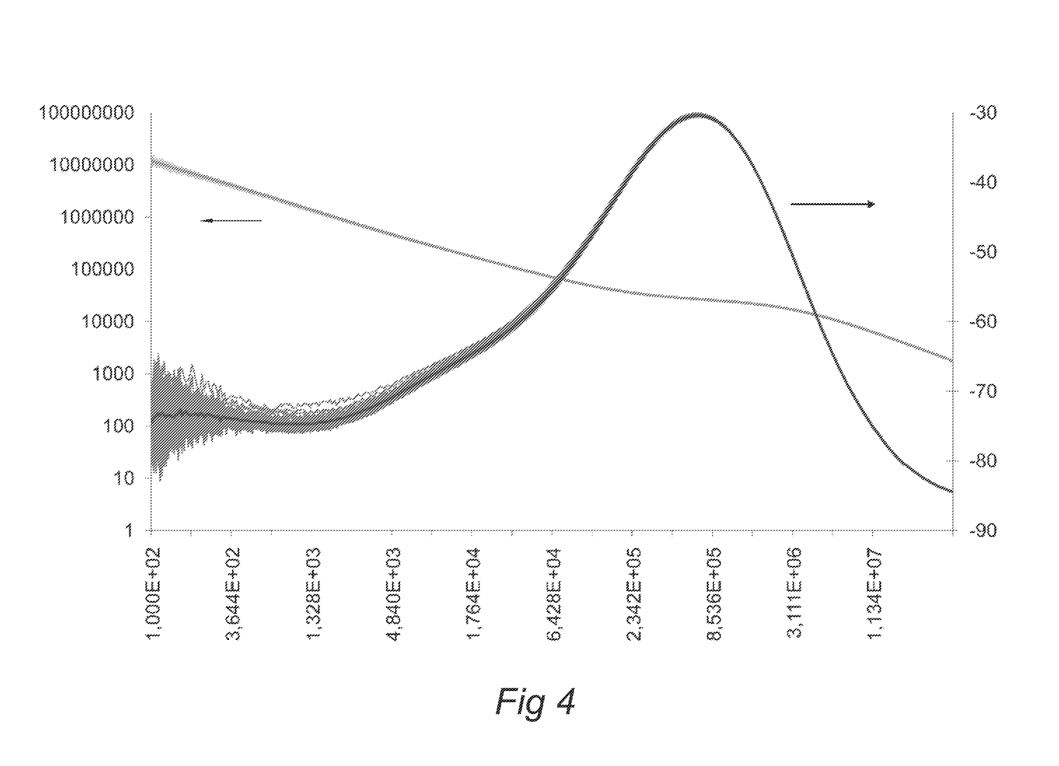

FIGS. 4-6 are further described below. FIGS. 7a-7c schematically depict a part of an alternative fabrication route. Assuming having reached the stage of FIG. 1a, an intermediate layer 300 is provided on the first first wafer side or top surface 211 of the first wafer 201. This intermediate layer 300 is especially a polymeric layer, such as PDMS, SU8, PMMA or PU, etc., and may especially have a height h in the range of 1-50 μm. In this intermediate layer 300, channel structure 140 is provided, in such a way that first electrode 110 and second electrode 120 are substantially free from the intermediate layer 300 and are comprised by the channel wall 141. Then, the system thus obtained is closed with a closure 400, which closure 400 comprises the floating electrode 130. For instance, the closure may be a wafer comprising the floating electrode 130. However, the closure itself may also be electrically conductive, and have the function of the floating electrode 130. For instance, the closure 400 may be a metal plate. Access holes, not depicted, may be provided in the closure 400 or in the second wafer 202, but preferably in the closure 400.

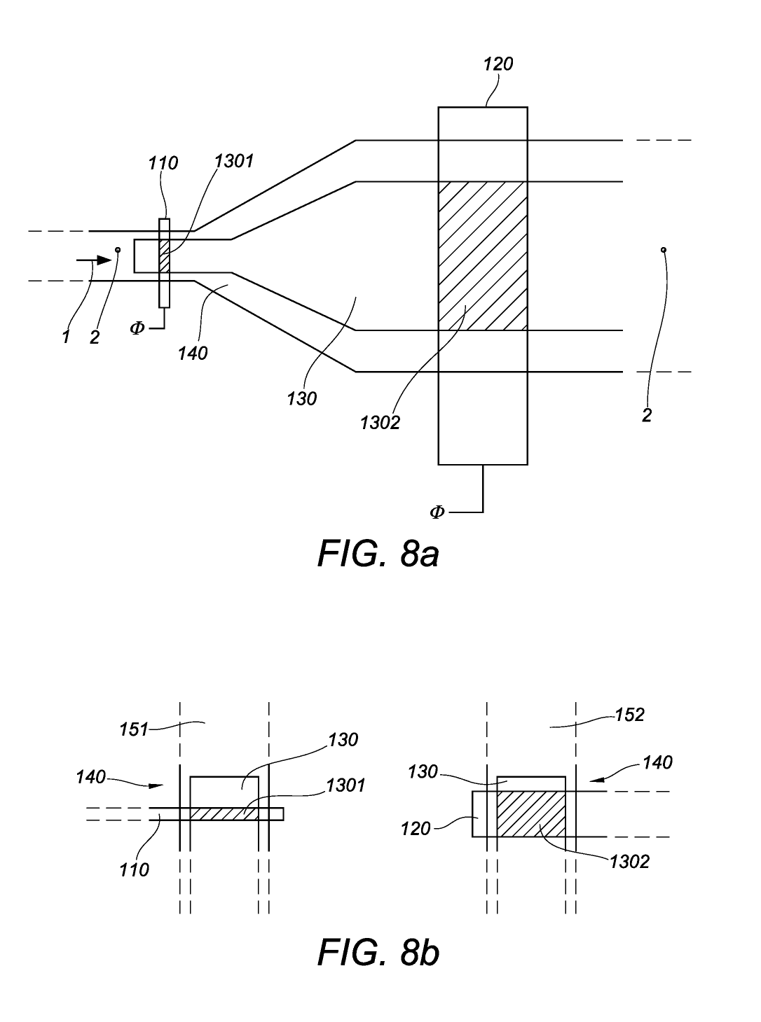

FIG. 8a schematically depicts an embodiment with a single channel 140. The first electrode 110 and the floating electrode 130 are arranged opposite of each other (and of course not in electrical contact with each other). Those parts that are directly facing each other have an overlapping area, indicated with reference 1301. Likewise, second electrode 120 and the floating electrode are arranged opposite of each other (and of course not in electrical contact with each other). Those parts that are directly facing each other have an overlapping area, indicated with reference 1302. The latter area is a number of times, such a about 10 times larger than the former area. In this way, a double count signal at 120/130 (i.e. at overlapping area 1302) of analyte 2 has no substantial influence on the total signal. FIG. 8a thus schematically depicts a single channel variant. The same principle may be applied for the multi-channel variant, as very schematically depicted in FIG. 8b.

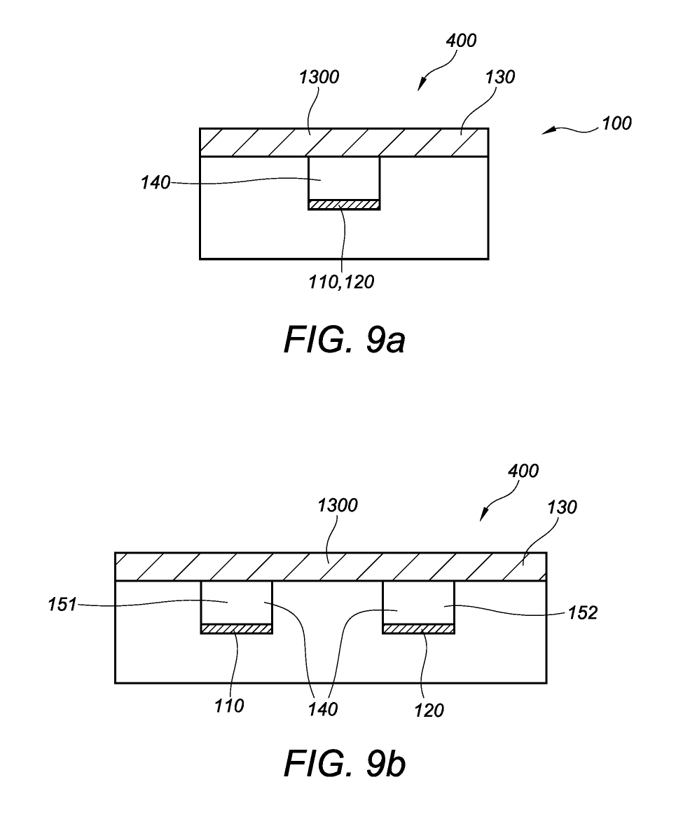

FIGS. 9a-9b schematically depict a specific embodiment of the floating electrode 130. The device 100 is shown in a cross-sectional view. Here, the floating electrode 130 is a plate like electrode, indicated with reference 1300, for instance covering a substantial part of the channel 140, such as in FIG. 9a, or both the first channel 151 and the second channel 152, such as in FIG. 9b. For instance, the closure 400 may be used as floating electrode. For instance, the floating electrode 130 may comprise a Si electrode or a metal plate. Referring to instance FIG. 3a or 7c, the wafer 201, or at least substantial part thereof, may be applied as floating electrode 130. Note that in FIG. 9a the reference 110,120 indicates here that the schematically depicted cross-section could be a cross-sectional view at the first electrode 110 as well as at the second electrode 120.

### EXAMPLES

### Method

The schematic diagram of the fabrication process is shown in FIGS. 1a-1i (see also above). Compared to the fabrication process for planar electrodes that was previously used, this process contains only one additional step. The micro fluidic chips were made of two 500 μm 4″ Borofloat glass substrates. In the top wafer the micro fluidic channels with floating electrodes were made. This substrate was covered with sputtered Cr and Au layers; the Cr layer functioned as adhesion layer for Au. This step was followed by a photolithography step and wet etching of the Cr and Au layers. Subsequently the micro fluidic channel was isotropically etched in a HF solution. Making the floating electrode is het additional step in the process. This electrode was made by placing a shadow mask on top of the photo resist, followed by sputtering of Pt forming the floating electrode. After that access holes were powder blasted from the backside using a photo patternable foil. On the bottom wafer Pt electrodes were placed, which are the connecting electrodes. These were prepared by etching a recess with buffered HF, after a photolithography step. The recess was filled with sputtered Pt (180 nm) with Ta as adhesion layer. At last the photo resist was removed, leaving a plane glass surface. This surface was bonded to the channel side of the top glass wafer using fusion bonding. After that it was diced into separate chips.

Four types of micro fluidic chips were designed. Each type of micro fluidic chips consists of a micro channel with a depth of 18 μm. Due to the floating electrode, the chip consists of two parallel electrode pairs. It is not desirable to have both electrode pairs in the same channels, since there is a probability that particle are simultaneously between both electrode pairs. Therefore the second electrode pair is separated from the first electrode pair in such a way that no particles flow between the second electrode pair. So the particles are only detected between the first electrode pair. For each of the four types, the micro channel tapers to a width of 38 μm at the electrode area of this first electrode pair. Since the floating electrode was made by using the photo resist mask of the micro fluidic channel, it has a width at this electrode area of 2 μm.

The differences in the four types are in the electrode area of the second electrode pair and if the type contains a filter system or an additional micro fluidic channel. In the table below the characteristics of each of the four types are shown. The electrode area of the second electrode pair can be the same as the first electrode pair or 20 times larger. Since the particles are measured between the first electrode pair, it is expected that the increase in the electrode area of the second electrode pair improves the sensitivity, since its impedance is decreased. Besides this difference, the type can also contain a filter or not. Due to this mechanical filter, the particles will not enter the channel with the second electrode pair, such that only the impedance of the background electrolyte is measured. When there is no filter, the types consist of two micro channels that are parallel to each other. One of the micro channels is filled with the suspension, while the other one has background electrolyte in it.

| Chip Type | Filter | Area of EP2 |
|---|---|---|
| 1A | Yes | Same as EP1 |
| 1B | No | Same as EP1 |
| 2A | Yes | 20 times larger than EP1 |
| 2B | No | 20 times larger than EP1 |

Besides the additional step in the fabrication process, one channel in the chip needs to be blocked before experiments can be done. The floating electrode is sputtered on the bottom of the channel, implicating that there needs to be a micro fluidic channel between the electrode pairs. As already mentioned, such connection is not desirable, since particles can be simultaneously between both pair. Therefore this channel is designed with an additional access hole, such that it can be filled with UV glue. For the gluing Loctite 358 was used and when exposed to UV radiation of 365 nm it cures. A drop of glue was put into the access hole and after several seconds, the UV source (ELC-403, Electro-lite Corporation) was turned on, causing the glue to cure.

### Measurement Set-Up

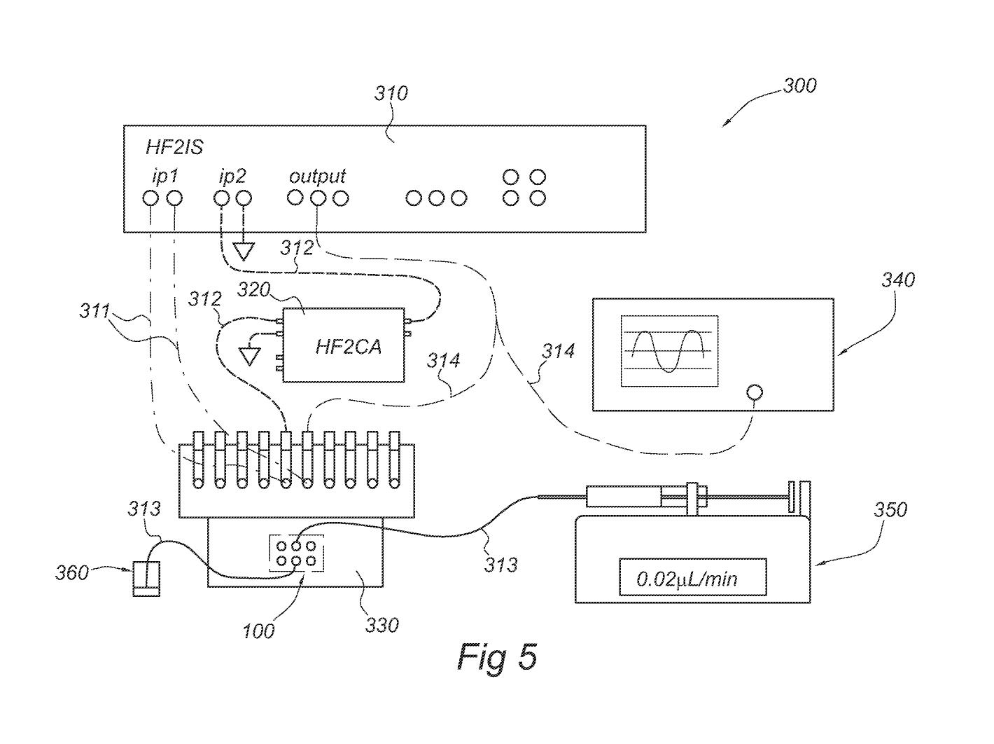

For all experiments the chip was put into a chip holder 330 (see also FIG. 5, schematically showing a measurement setup 300 used), such that reliable fluidic and electrical connections could be made. The chip holder contains screw threads which are aligned with the access holes of the micro fluidic chip. Using a pump 350, here Harvard PHD2000 syringe pump, the fluid was pumped through the chip via tube 313, here a glass capillary (inner diameter 148 μm) and connected to the micro fluidic chip 100 using Upchurch nuts and ferrules (Upchurch Scientific).

Two types of experiments were performed. The first study involves the measurement of the frequency characteristics of the different types of the micro fluidic chip. For this purpose the chips were filled with background electrolyte and a bode plot from 100 Hz to 40 MHz was made using a HP impedance/gainphase analyzer type HP4194A, controlled by LabVIEW (7 Express, version 7.0, 2003, National Instruments).

In the second study beads suspended in the background electrolyte were detected using electrical impedance measurements. From the results of the first study, the optimal measurement frequencies for the electrical impedance measurements were determined. The electrical impedance was measured using an impedance spectroscope 310, here HF2IS impedance spectroscope, in combination with a current amplifier 320, here HF2CA current amplifier (both Zurich Instruments, Zurich, Switzerland). In FIG. 5 a schematic diagram of the electrical impedance measurement is shown. The HF2IS impedance spectroscope was used to generate the excitation signal 314 (1 VPP, 600 kHz) as well as measurement of the voltages at two inputs. An oscilloscope (Agilent Technologies, type DS03062A) 340 was connected to the impedance spectroscope to ensure that the excitation signal is right. The first input measured the voltage 311 over the micro fluidic chip, while the other input measured the signal from the HF2CA current amplifier and indirectly thus the current 312 trough the micro fluidic chip. So a four point measurement was done with this configuration. Both input signals were captured on a laptop and used for analysis using Matlab (R2007B, version 7.5.0.342, 2007, the Mathworks Inc). In Matlab the electrical impedance was calculated from the two inputs. After that the program was used for the calculation of the peak heights in the same manner as described in previous work.

Reference 360 indicates the receipt of waste, for instance escaping from the first channel and/or second channel through access channel(s).

### Samples

Two sizes of polystyrene beads were used during the experiments. These are Polybead Polystyrene Blue Dyed beads with a diameter of 6 μm and Polybead Polystyrene Red Dyed beads with a diameter of 3 μm, both obtained from Polysciences Inc (Warrington, Pa. USA). The beads were suspended in Ferticult™ Flushing medium (chemically balanced salt solution, HEPES buffered with 0.4% HSA, purchased from Fertipro NV (Beernem, Belgium)) with a specific electrical conductivity of 1.4 S·m⁻¹.

### Results

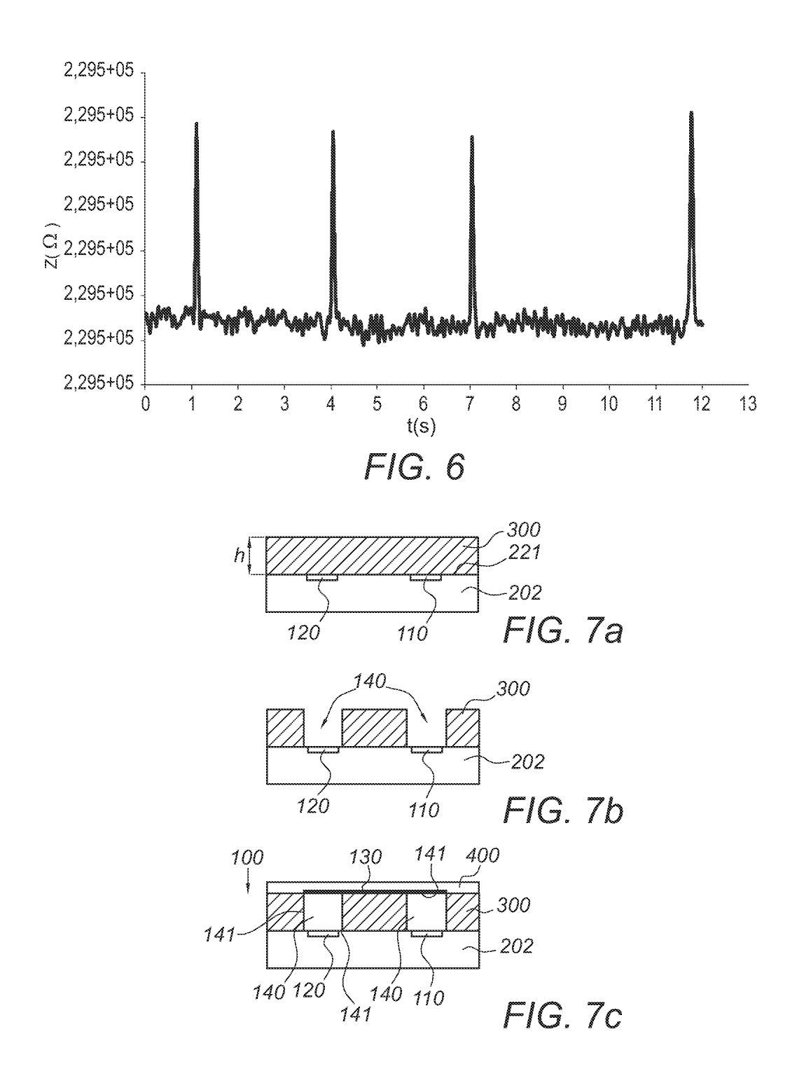

A new process is developed for the fabrication of parallel electrodes in a micro channel (FIGS. 1a-1i). The step in FIG. 1c is the extra necessary step compared to our fabrication process for planar electrodes. In order to operate the chip with floating electrode properly, the chip consists of two electrode pairs (FIGS. 2a-2e and 3b-3c) separated from each other since otherwise particles could be simultaneously detected. UV glue is used for the blocking The frequency behaviour of the lab-on-a-chip device filled with washing medium was first determined (FIG. 4). Finally, 6 μm (standard deviation is 0.477 μm) polystyrene beads suspended in washing medium were guided along electrode pair 1 and detected with electrical impedance measurements at 600 kHz (461±124Ω for 95 beads, FIG. 6). The relative deviation in the impedance change is identical to the size distribution of the beads.

1. A lab-on-a-chip device comprising a micro channel for a fluid, the micro channel comprising a first micro channel and a second micro channel, wherein the micro channel comprises a circumferential channel wall with the first micro channel comprising a circumferential first channel wall and the second micro channel comprising a circumferential second channel wall, wherein the channel wall comprises a first electrode, a second electrode, and a floating third electrode, wherein the first channel wall comprises the first electrode, the second channel wall comprises the second electrode, and wherein both the first channel wall and the second channel wall comprise the floating third electrode.

2. The lab-on-a-chip device according to claim 1, wherein at locations of the first and the second electrode the micro channel has a channel width and channel height in the range of 0.1-500 μm, especially in the range of 5-50 μm, preferably wherein at the locations of the first and the second electrode the micro channel has a channel width in the range of 1-500 μm, and a channel height in the range of 0.1-300 μm, especially a channel width and channel height in the range of 2-200 μm.

3. The lab-on-a-chip device according to claim 1, wherein the first electrode and the second electrode are arranged opposite of the floating third electrode.

4. The lab-on-a-chip device according to claim 1, wherein the device comprises a layered structure comprising a first wafer, comprising the micro channel, and comprising the floating electrode, and a second wafer, comprising the first electrode and the second electrode, wherein the first wafer and the second wafer are configured relative to each other to provide the micro channel with the circumferential channel wall, wherein the channel wall comprises the first electrode, the second electrode, and the floating third electrode.

5. The lab-on-a-chip device according to claim 1, wherein the device comprises a layered structure comprising a first wafer, comprising the floating electrode, an intermediate layer comprising the micro channel, and a second wafer, comprising the first electrode and the second electrode, wherein the first wafer, the intermediate layer and the second wafer are configured relative to each other to provide the micro channel with the circumferential channel wall, wherein the channel wall comprises the first electrode, the second electrode, and the floating third electrode.

6. The lab-on-a-chip device according to claim 1, wherein the first micro channel and the second micro channel are not in fluid connection which each other.

7. The lab-on-a-chip device according to claim 1, wherein the first micro channel and the second micro channel are in fluid connection which each other, and wherein a fluid permeable membrane is arranged between the first micro channel and the second micro channel.

8. The lab-on-a-chip device according to claim 1, wherein the first micro channel and the second micro channel are interconnected by an interconnecting channel, wherein the interconnecting channel is configured to host at least part of the floating third electrode, and wherein the interconnecting channel further comprises a fluid barrier.

9. The lab-on-a-chip device according to claim 1, wherein the first electrode in the first micro channel has a first electrode surface area, wherein the second electrode in the second micro channel has a second electrode surface area, wherein the floating electrode has in the first micro channel a floating electrode first surface area and in the second micro channel a floating electrode second surface area, and wherein one or more of (1) the second electrode surface area is larger than the first electrode surface area, and (2) the floating electrode second surface area is larger than the floating electrode first surface area, applies.

10. A lab-on-a-chip device comprising a micro channel for a fluid, wherein the micro channel comprises a circumferential channel wall, wherein the channel wall comprises a first electrode, a second electrode, and a floating third electrode.

11. The lab-on-a-chip device according to claim 1 wherein the first electrode and the floating electrode have an overlapping area where they are facing each other, wherein the second electrode and the floating electrode have an overlapping area where they are facing each other, and wherein the overlapping area between the second electrode and the floating electrode is at least 10 times the overlapping area between the first electrode and the floating electrode.

12. An electronic lab-on-a-chip device comprising the lab-on-a-chip device according to claim 1 and an electronic device, electronically connected with the first and the second electrode, wherein the electronic device is selected from the group consisting of a current meter, a voltmeter, a current generator, a voltage source, and an electrical impedance meter, and wherein the electronic device especially comprises an electrical impedance meter configured to measure the electrical impedance between the first electrode and the second electrode.

13. A method for the analysis of an analyte fluid comprising flowing the analyte fluid through the channel of the electronic lab-on-a-chip device according to claim 12 and measuring an electric signal between the first and the second electrode, especially measuring the electrical impedance between the first electrode and the second electrode.

14. Use of the electronic lab-on-a-chip device according to claim 12, for the analysis of mammalian semen.

15. A method for the production of a lab-on-a-chip device according to claim 1, comprising:
a. providing a first wafer, with a first first wafer side and an opposite second first wafer side, and a second wafer, with a first second wafer side and an opposite second second wafer side;
b. creating a micro channel structure in the first first wafer side;
c. providing a floating electrode to a wall of the micro channel structure;
d. creating at least a first access channel and a second access channel between the micro channel structure and the second first wafer side;
e. providing a first electrode and a second electrode to the first second wafer side;
f. attaching the first first wafer side and the first second wafer side to each other with the first electrode and second electrode comprised by the micro channel structure.

16. A method for the production of a lab-on-a-chip device according to claim 1, comprising:
a. providing a first wafer, with a first first wafer side and an opposite second first wafer side, and a closure, with a first closure side and an opposite second closure side, wherein the first closure side comprises a floating electrode;
b. creating at least a first access channel and a second access channel between the first closure side and the opposite second closure side;
c. providing a first electrode and a second electrode to the first second wafer side;
d. providing an intermediate layer to the first second wafer side with first electrode and second electrode;
e. creating a micro channel structure in the intermediate layer, with the first electrode and the second electrode comprised by the micro channel structure;
f. providing the intermediate layer and the first closure side to each other with the floating electrode comprised by the micro channel structure.

17. A method for the production of a lab-on-a-chip device according to claim 1, comprising:
a. providing a first wafer, with a first first wafer side and an opposite second first wafer side, and a second wafer, with a first second wafer side and an opposite second second wafer side;
b. creating a micro channel structure in the first first wafer side;
c. providing a floating electrode to a wall of the micro channel structure;
d. providing a first electrode and a second electrode to the first second wafer side;
e. attaching the first first wafer side and the first second wafer side to each other with the first electrode and second electrode comprised by the micro channel structure.

18. The method according to claim 15, wherein the first electrode and the floating electrode have an overlapping area where they are facing each other, wherein the second electrode and the floating electrode have an overlapping area where they are facing each other, and wherein the overlapping area between the second electrode and the floating electrode is at least 10 times the overlapping area between the first electrode and the floating electrode.
